(19) United States
(12) Patent Application Publication
(10) Pub. No.: US 2018/0192601 A1
(43) Pub. Date: Jul. 12, 2018

(54) Composition and Process for Producing Plants Using Anthers of Jatropha Species

(71) Applicant: Reliance Industries Limited, Mumbai (IN)

(72) Inventors: Savarimuthu AROCKIASAMY, Hyderadad (IN); Neeta Shailesh MADAN, Mumbai (IN); Sainath HADOLE, Samalkota (IN); Janyavula Venkata NARASIMHAM, Hyderabad (IN)

(73) Assignee: Reliance Industries Limited, Mumbai (IN)

(21) Appl. No.: 15/866,771

(22) Filed: Jan. 10, 2018

(30) Foreign Application Priority Data
Jan. 10, 2017 (IN) 201721001042

(51) Int. Cl. A01H 4/00; A01H 5/02; C12N 5/00

(52) U.S. Cl. CPC A01H 4/005; A01H 4/008; C12N 5/0068; C12N 5/0025; A01H 5/02

(57) ABSTRACT

The present invention describes a composition for developmental stages including but not limited to callus induction, proliferation, somatic embryogenesis, somatic embryo maturation and embryo germination, of Jatropha plants. The disclosure further describes a process of producing plants of Jatropha species from anthers, wherein the Jatropha species includes but is not limited to haploid plants, double haploid plants, tetraploid plants or polyploid plants by employing the said composition.

### TECHNICAL FIELD

The present disclosure describes a composition, particularly to a media composition. The disclosure further describes a process of producing plants of Jatropha species from anther, wherein the Jatropha species includes but is not limited to haploid plants, double haploid plants, tetraploid plants and polyploid plants.

### BACKGROUND OF THE DISCLOSURE

The breeding programs in Jatropha are long reproductive cycle with a juvenile phase that lasts several months, the highly heterozygous nature of the genome, the large canopy size, and self-incompatibility which is a long-term process requires multiple cycles of self-pollination to achieve complete homozygosity, complexing the process of producing Jatropha. Obtaining homozygous Jatropha species generally takes about 8 to 10 generations in traditional breeding with the timeline of about 4 years to 6 years. Various biotechnology techniques provide tools for plant breeding such as tissue culture, wherein haploid and doubled haploid production leads to homozygosity. However, the efficiency of developing a haploid and doubled haploid Jatropha by the biotechnology techniques has very low efficiency. Further, Jatropha is a recalcitrant crop to tissue cultures and transformation, which adds to the low efficiency observed in their development. It is observed that use of latex or phenolics during Jatropha tissue culture leads to browning and necrosis, leading to arrest of cell proliferation.

Therefore, there appears to be a need to overcome the limitations in developing haploid, double haploid or polyploid plants of Jatropha.

### STATEMENT OF THE DISCLOSURE

The present disclosure describes a composition, particularly a media composition comprising Murashige and Skoog (MS) macro elements, MS micro elements, vitamins, binder, buffer, carbohydrate, gelling agent, optionally along with component selected from a group comprising growth regulator, additive and amino acid, or any combinations thereof.

The present disclosure further describes a process for preparing the said composition.

The present disclosure furthermore describes a process for producing plants of Jatropha species from anthers, wherein the Jatropha species includes but is not limited to haploid plants, double haploid plants, tetraploid plants or polypoid plants. In an embodiment, the said process of producing the plants of Jatropha species employs the composition of the present disclosure.

### BRIEF DESCRIPTION OF ACCOMPANYING FIGURES

The patent or application file contains at least one drawing executed in color. Copies of this patent with color drawings will be provided by the Patent and Trademark Office upon request and payment of the necessary fees.

In order that the disclosure may be readily understood and put into practical effect, reference will now be made to exemplary embodiments as illustrated with reference to the accompanying figures. The figures together with detailed description below, are incorporated in and form part of the specification, and serve to further illustrate the embodiments and explain various principles and advantages, in accordance with the present disclosure where:

FIG. 1 illustrates anther culture of Jatropha species as per the present disclosure.

FIG. 2 illustrates optical microcopy of Jatropha buds defining uni-nucleate stage of anthers.

FIG. 3 illustrates callus initiation from anthers of Jatropha in the composition of the present disclosure.

FIG. 4 illustrates regeneration of shoots of Jatropha with roots on the composition of the present disclosure.

FIG. 5 illustrates ploidy analysis of Control Jatropha Diploid sample.

FIG. 6 illustrates ploidy analysis of embryos/tissues showing haploid DNA peak.

FIG. 7 illustrates ploidy analysis of embryogenic calli/tissues developed in the present invention, showing haploid and dihaploid DNA peaks.

FIG. 8 illustrates confirmation of haploid and dihaploidy embryogenic calli or tissues of plants developed as per the present invention, using SSR markers.

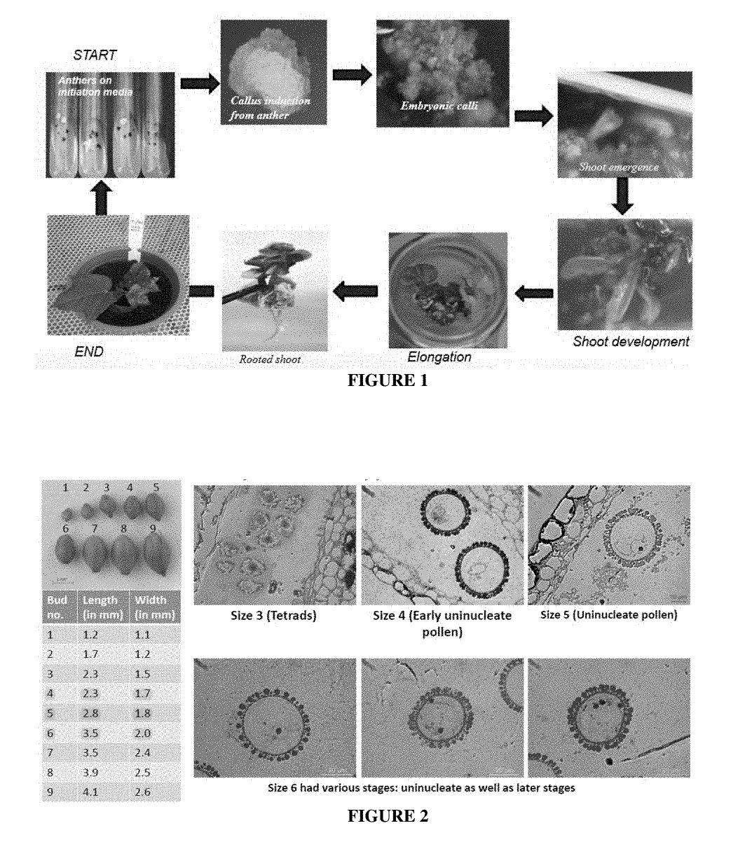

### DETAILED DESCRIPTION

The source and geographical origin of the Jatropha plants employed in the present disclosure is: Agroenergia De Honduras S.A. (AGROENHSA), Tegucigalpa Honduras, C.A., Honduras.

Further, the accession numbers of the Jatropha plants employed in the disclosure is provided below—

| Accession No. | Common Name | Crop Name |
|---|---|---|
| EC831436 | Jatropha | Jatropha Curcas |
| EC831437 | Jatropha | Jatropha Curcas |

The present disclosure describes a composition, particularly a media composition.

In an embodiment, the composition comprises MS macro elements, MS micro elements, vitamins, binder, buffer, carbohydrate, sugar alcohol, antioxidant, gelling agent, optionally along with component selected from a group comprising growth regulator, additive and amino acid, or any combinations thereof.

In an embodiment, the composition of the present disclosure comprises MS macro elements, MS micro elements, vitamins, buffer, binder, antioxidant, sugar alcohol, carbohydrate, additive, growth regulator and gelling agent.

In another embodiment, the composition of the present disclosure comprises MS macro elements, MS micro elements, vitamins, buffer, binder, antioxidants, sugar alcohol, carbohydrate, growth regulators and gelling agent.

In another embodiment, the composition of the present disclosure comprises MS macro elements, MS micro elements, vitamins, buffer, binder, antioxidants, sugar alcohol, carbohydrate and gelling agent.

In another embodiment, the composition of the present disclosure comprises MS macro elements, MS micro elements, vitamins, buffer, binder, antioxidants, sugar alcohol, carbohydrate, amino acid, additive, growth regulator and gelling agent.

In an embodiment, the MS macro elements in the composition is selected from a group comprising ammonium nitrate, potassium nitrate, calcium chloride dehydrate, magnesium sulfate heptahydrate and monopotassium phosphate, or any combinations thereof.

In an embodiment, the MS micro element in the composition is selected from a group comprising potassium iodide, dihydrogen borate, manganese sulfate tetrahydrate, zinc sulfate heptahydrate, sodium molybdate dehydrate, copper sulfate pentahydrate, cobalt chloride hexahydrate, ethylene diamine tetra acetic acid disodium salt dihydrate and ferrous sulfate heptahydrate, or any combinations thereof.

In an embodiment, the vitamin in the composition is selected from a group comprising inositol, thiamine, pyridoxine HCl and nicotinic acid, or any combinations thereof.

In an embodiment, the vitamin in the composition is MS vitamins or B5 vitamins.

In an embodiment, the binder in the composition is selected from a group comprising polyvinyl pyrrolidone (PVP), and charcoal, or a combination thereof.

In an embodiment, the buffer in the composition is selected from a group comprising MES (2-(N-morpholine) ethane sulfonic acid), and MOPS (3-(N-morpholino) propane sulfonic acid), or a combination thereof.

In an embodiment, the carbohydrate in the composition is selected from a group comprising maltose, sucrose, glucose and fructose, or any combinations thereof.

In an embodiment, the sugar alcohol in the composition is selected from a group comprising sorbitol and myo-inositol, or a combination thereof.

In an embodiment, the antioxidant in the composition is selected from a group comprising citric acid, ascorbic acid and DTT (Dithiothreitol), or a combination thereof.

In an embodiment, the additive in the composition is selected from a group comprising casein hydrolysate, coconut milk, malt extract and yeast extract, or any combinations thereof.

In an embodiment, the amino acid in the composition is selected from a group comprising proline, glycine, serine, glutamine and alanine, or any combinations thereof.

In an embodiment, the growth regulator in the composition is selected from a group comprising 2,4-dichloro phenoxy acetic acid (2,4-D), benzyl amino purine (BA), kinetin (6-furfuryl amino purine), indole-3-acetic acid, indole butyric acid, phloroglucinol and gibberellic acid, or any combinations thereof.

In an embodiment, the gelling agent in the composition is selected from a group comprising agar, phytagel and gellan gum, or a combination thereof.

In an embodiment, the MS macro element in the composition is at a concentration ranging from about 10% v/v to 20% v/v with respect to 10×MS macro element stock concentration.

In another embodiment, the MS macro element in the composition is in an amount ranging from about 100 ml to 200 ml of 10× stock concentration.

In another embodiment, the MS macro element in the composition is at a concentration of about 10% v/v, about 11% v/v, about 12% v/v, about 13% v/v, about 14% v/v, about 15% v/v, about 16% v/v, about 17% v/v, about 18% v/v, about 19% v/v or about 20% v/v, with respect to 10×MS macro element stock concentration.

In an embodiment, the MS micro element in the composition is at a concentration ranging from about 1% v/v to 5% v/v with respect to 100×MS micro element stock concentration.

In another embodiment, the MS micro element in the composition is in an amount ranging from about 10 ml to 50 m of 100× stock concentration.

In another embodiment, the MS micro element in the composition is at a concentration of about 1% v/v, about 1.5% v/v, about 2% v/v, about 2.5% v/v, about 3% v/v, about 3.5% v/v, about 4% v/v, about 4.5% v/v or about 5% v/v, with respect to 100×MS micro element stock concentration.

In an embodiment, the vitamin in the composition is at a concentration ranging from about 0.5% v/v to 1% v/v with respect to 200×MS vitamin stock concentration.

In another embodiment, the vitamin the composition is at a concentration of about 0.5% v/v, about 0.6% v/v about 0.7% v/v, about 0.8% v/v, about 0.9% v/v or about 1.0% v/v, with respect to 200×MS vitamin stock concentration.

In another embodiment, the vitamin in the composition is in an amount ranging from about 5 ml to 20 ml of 200× stock concentration.

In an embodiment, Table A illustrates the stock concentrations of MS macro element, MS micro element and vitamins employed in the composition of the present disclosure.

TABLE A

| | Quantity (mgl- | Stock |
|---|---|---|
| Components | 1) | concentration |
| Macronutrients | 1X | |
| | concentration | |
| NH4NO3 | 1650 | 10X |
| KNO3 | 1900 | |
| CaCI22H2O | 440 | |
| MgSO4•7H2O | 370 | |
| KH2PO4 | 170 | |
| Micronutrients | | |
| KI | 0.83 | 100X |
| H2BO3 | 6.2 | |
| MnSO4•4H2O | 22.3 | |
| ZnSO47H2O | 8.6 | |
| Na2MoO4•2H2O | 0.25 | |
| CuSO45H2O | 0.025 | |
| CoCI2•6H2O | 0.025 | |
| Na2EDTA | 37.3 | |
| FeSO4•7H2O | 27.8 | |
| Vitamins | 1X | |
| | concentration | |
| Inositol | 100 | 200X |
| Glycine | 2 | |
| Thiamine | 0.1 | |
| Pyridoxine HCI | 0.5 | |
| Nicotinic acid | 0.5 | |

In an embodiment, the binder in the composition is at a concentration ranging from about 0.025% w/v to 0.05% w/v.

In another embodiment, the binder in the composition is at a concentration of about 0.025% w/v, about 0.027% w/v, about 0.029% w/v, about 0.031% w/v, about 0.033% w/v, about 0.035% w/v, about 0.037% w/v, about 0.039% w/v, about 0.041% w/v, about 0.043% w/v, about 0.045% w/v, about 0.046% w/v, about 0.047% w/v, about 0.048% w/v, about 0.049% or about 0.050% w/v.

In an embodiment, the antioxidants in the composition is at a concentration ranging from about 0.003% w/v to 0.006% w/v.

In another embodiment, the antioxidants in the composition is at a concentration of about 0.003% w/v, about 0.0032% w/v, about 0.0034% w/v, about 0.0036% w/v, about 0.0038% w/v, about 0.004% w/v, about 0.0042% w/v, about 0.0044% w/v, about 0.0046% w/v, about 0.0048% w/v, about 0.005% w/v, about 0.0052% w/v, about 0.0054% w/v, about 0.0056% w/v, about 0.0058% w/v or about 0.006% w/v.

In an embodiment, the additive in the composition is at a concentration ranging from about 0.03% w/v to 0.05% w/v.

In another embodiment, the additive in the composition is at a concentration of about 0.03%, about 0.032% w/v, about 0.034% w/v, about 0.036% w/v, about 0.038% w/v, about 0.04% w/v, about 0.042% w/v, about 0.044% w/v, about 0.046% w/v, about 0.048% w/v or about 0.05% w/v.

In an embodiment, the carbohydrate in the composition is at a concentration ranging from about 1% w/v to 9% w/v.

In another embodiment, the carbohydrate in the composition is at a concentration of about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v or about 9% w/v.

In an embodiment, the sugar alcohol in the composition is at a concentration ranging from about 0.01% w/v to 0.04% w/v.

In another embodiment, the sugar alcohol in the composition is at a concentration of about 0.01% w/v, about 0.012% w/v, about 0.014% w/v, about 0.016% w/v, about 0.018% w/v, about 0.02% w/v, about 0.022% w/v, about 0.024% w/v, about 0.026% w/v, about 0.028% w/v, about 0.03% w/v, about 0.032% w/v, about 0.034% w/v, about 0.036% w/v, about 0.038% w/v or about 0.04% w/v.

In an embodiment, the buffer in the composition is at a concentration ranging from about 0.005% w/v to 0.05% w/v.

In another embodiment, the antioxidant in the composition is at a concentration of about 0.005% w/v, about 0.006% w/v, about 0.007% w/v, about 0.008% w/v, about 0.009% w/v, about 0.01% w/v, about 0.011% w/v, about 0.012% w/v, about 0.013% w/v, about 0.014% w/v, about 0.015% w/v, about 0.016% w/v, about 0.017% w/v, about 0.018% w/v, about 0.019% w/v or about 0.05% w/v.

In an embodiment, the amino acid is at a concentration ranging from about 0.05% w/v to 0.1% w/v.

In another embodiment, the amino acid is at a concentration of about 0.05%, about 0.06%, about 0.07% w/v, about 0.08% w/v, about 0.09% w/v or about 0.1% w/v.

In an embodiment, the growth regulator in the composition is at a concentration ranging from about 0.00001% w/v to 0.0005% w/v.

In another embodiment, the growth regulator in the composition is at a concentration of about 0.00001% w/v, about 0.00002% w/v, about 0.00003% w/v, about 0.00004% w/v or about 0.00005% w/v.

In an embodiment, the gelling agent in the composition is at a concentration ranging from about 0.4% w/v to 1% w/v.

In another embodiment, the gelling agent in the composition is at a concentration of about 0.4% w/v, about 0.42% w/v, about 0.44% w/v, about 0.46% w/v, about 0.48% w/v, about 0.5% w/v, about 0.52% w/v, about 0.54% w/v, about 0.56% w/v, about 0.58% w/v, about 0.6% w/v, about 0.62% w/v, about 0.64% w/v, about 0.66% w/v, about 0.68% w/v, about 0.7% w/v, about 0.72% w/v, about 0.74% w/v, about 0.76% w/v, about 0.78% w/v, about 0.8% w/v, about 0.82% w/v, about 0.84% w/v, about 0.86% w/v, about 0.88% w/v, about 0.9% w/v, about 0.92% w/v, about 0.94% w/v, about 0.96% w/v, about 0.98% w/v or about 1% w/v.

In an embodiment, the composition of the present disclosure comprises MS macro elements, MS micro elements, MS vitamins, citric acid, Polyvinyl pyrrolidone (PVP), ascorbic acid, myo inositol, maltose, casein hydrdolysate, 2,4-Dichloro phenoxy acetic acid (2,4-D), benzyl amino purine (BA), kinetin (6-furfuryl amino purine) and agar.

In another embodiment, the composition of the present disclosure comprises MS macro elements, MS micro elements, B5 vitamins, citric acid, polyvinyl pyrrolidone (PVP), ascorbic acid, myo inositol, sucrose, indole-3-acetic acid (IAA), benzyl amino purine (BA), kinetin (6-furfurly amino purine) and agar.

In another embodiment, the composition of the present disclosure comprises MS macro elements, MS micro elements, MS vitamins, citric acid, polyvinyl pyrrolidone (PVP), ascorbic acid, myo inositol, maltose and phytagel.

In another embodiment, the composition of the present disclosure comprises MS macro elements, MS micro elements, MS vitamins, citric acid, polyvinyl pyrrolidone (PVP), ascorbic acid, myo inositol, maltose, proline, casein hydrolysate, sorbitol, benzyl amino purine (BA), kinetin (6-furfurly amino purine), indole butyric acid and phytagel.

In another embodiment, the composition of the present disclosure comprises MS macro elements, MS micro elements, MS vitamins, citric acid, polyvinyl pyrrolidone, ascorbic acid, myo inositol, sucrose, indole butyric acid, phloroglucinol, gibbrellic acid and agar.

In an embodiment, the pH of the composition is ranging from about 5 to 6.

In another embodiment, the pH of the composition is about 5.0, about 5.1, about 5.2, about 5.3, about 5.4, about 5.5, about 5.6, about 5.7, about 5.8, about 5.9 or about 6.0.

In an embodiment, the composition of the present disclosure causes at least one of callus induction, proliferation, somatic embryogenesis, somatic embryo maturation, embryo germination, or any combination thereof, of Jatropha species.

In another embodiment, the composition of the present disclosure causes callus induction, proliferation, somatic embryogenesis, somatic embryo maturation and embryo germination, of Jatropha species

In an embodiment, the composition causes about 70% to 100% callus induction by anther culture of Jatropha species.

In another embodiment, the composition causes about 70% callus induction, about 75% callus induction, about 80% of callus induction, about 85% callus induction, about 90% callus induction, about 95% callus induction or about 100% callus induction, by anther culture of Jatropha species.

In another embodiment, the composition causes 100% callus induction by anther culture of Jatropha species.

In another embodiment, the composition causes high frequency callus initiation inducting 100% callus from anthers of Jatropha species.

In an embodiment, the composition of the present disclosure causes development of plants of Jatropha species including but not limiting to haploid plant, double haploid plant, tetraploid plant or polyploid plant of Jatropha, through callus induction.

In an embodiment, the composition causes regeneration of shoot comprising roots, in Jatropha, from anther derived callus.

In an embodiment, the composition of the present disclosure causes embryogenic calli initiation after callus induction, within a period of about 8 weeks to 12 weeks.

In an embodiment, the composition of the present disclosure causes development of Jatropha plants including but is not limited to haploid plant, double haploid plant and polyploid plant from anthers in a period of about 8 months to 12 months from the time of initial cross, unlike the conventional breeding which takes about 4 years to 6 years to achieve homozygosity.

The present disclosure describes a process of preparing the composition of the present disclosure.

In an embodiment, the process preparing the composition comprises step of dissolving MS macro elements, MS micro elements, vitamins, buffer, binder, antioxidant, sugar alcohol carbohydrate, gelling agent, optionally along with component selected from a group comprising growth regulator, additive and amino acid, or any combination thereof, in a solvent including but is not limited to water and milliQ water.

In an embodiment, the process of preparing the composition comprises dissolving the components of the composition in a specific sequence, in milliQ water.

In an embodiment, the process of preparing the composition comprises—
dissolving MS macro elements, MS micro elements, MS vitamins, citric acid, polyvinyl pyrrolidone (PVP), ascorbic acid, myo inositol, maltose, casein hydrolysate, 2,4-dichloro phenoxy acetic acid (2,4-D), benzyl amino purine (BA) and kinetin (6-furfurly amino purine), in milliQ water; and
adjusting the pH with potassium hydroxide, followed by adding agar to obtain the composition.

In another embodiment, the process of preparing the composition comprises—
dissolving MS macro elements, MS micro elements, B5 vitamins, citric acid, PVP, ascorbic acid, myo inositol, sucrose, indole-3-acetic acid, benzyl amino purine and kinetin, in milliQ water;
adjusting the pH with potassium hydroxide, followed by adding agar to obtain the composition.

In another embodiment, the process of preparing the composition comprises—
dissolving MS macro elements, MS micro elements, MS vitamins, citric acid, PVP, ascorbic acid, myo inositol and maltose, in milliQ water;
adjusting the pH with potassium hydroxide, followed by adding gellan gum to obtain the composition.

In another embodiment, the process of preparing the composition comprises—
dissolving MS macro elements, MS micro elements, MS vitamins, citric acid, PVP, ascorbic acid, myo inositol, maltose, proline, casein hydrolysate, sorbitol, benzyl amino purine, kinetin and indole butyric acid, in milliQ water;
adjusting the pH with potassium hydroxide, followed by adding phytagel to obtain the composition.

In another embodiment, the process of preparing the composition comprises—
dissolving MS macro elements, MS micro elements, MS vitamins, citric acid, PVP, ascorbic acid, myo inositol, sucrose, indole butyric acid, phloroglucinol and gibrellic acid, in milliQ water;
adjusting the pH with potassium hydroxide, followed by adding agar to obtain the composition.

In an embodiment, during the process of preparing the composition the pH of the composition is maintained at about 5.6, about 5.7 or about 5.8 with the aid of base including but is not limited to potassium hydroxide.

In an embodiment, the process of preparing the composition comprising making up the volume of the composition with solvent including but is not limited to water and milliQ water.

The present disclosure further describes a process of producing plants of Jatropha species.

In an embodiment, the present disclosure describes a process for producing plants of Jatropha species including but not limiting to haploid plant, double haploid plant, tetraploid plant and polyploid Jatropha, employing the composition described above.

In an embodiment, the process of the present disclosure for producing haploid plant, double haploid plant, tetraploid plant or polyploid of Jatropha species through anthers is a high throughput process.

In an embodiment, the process of the present disclosure produces plants of Jatropha species including but not limiting to Jatropha curcus and Jatropha integerrima, through anthers, employing the composition described above.

In an embodiment, the process of the present disclosure causes progenies of inter species and back crosses for accelerated breeding program in Jatropha species by employing the composition described above.

In an embodiment, the process of producing Jatropha plants including but not limiting to haploid plant, double haploid plant, tetraploid plant and polyploid plant comprises steps of:
growing donor plants;
collecting immature flowers from the donor plants, followed by subjecting the flowers to physical treatment and sterilization;
extracting the anthers form the flowers, followed by inoculating the anther in the composition described above;
inducing callus formation, followed by proliferation; and
inducing somatic embryogenesis, followed by germination, thereby producing Jatropha plants.

In an embodiment, the step of collecting immature flowers from the donor plants and physical treatment and sterilization of the said flowers comprises:
collecting healthy and disease-free inflorescence of Jatropha lines from the field;
washing the inflorescence in water and then treating with water containing detergent solution;
washing the inflorescence in water for about 5 minutes to 10 minutes, followed by treating the inflorescence with about 0.1% to 0.5% Bavistin and washing with milliQ water for about 4 times to 6 times;
surface sterilizing the inflorescence by immersing in about 70% v/v ethanol for about 1 minutes to 3 minutes; and
sterilizing the inflorescence with about 50% v/v sodium hypochlorite for about 15 minutes containing tween 20, followed by rinsing in sterile water for about 5 times to 6 times.

In an embodiment, after sterilization, the inflorescence is incubated at a temperature ranging from about 4° C. to 8° C. for a period ranging from about 4 days to 7 days in a closed air tight container under aseptic condition.

In an embodiment, post incubation at temperature ranging from about 4° C. to 8° C., the flower buds of about 1 mm to 2.5 mm are selected for excision of anthers. The flower buds are blot dried with several layers of sterile filter papers to remove excess moisture before the excision.

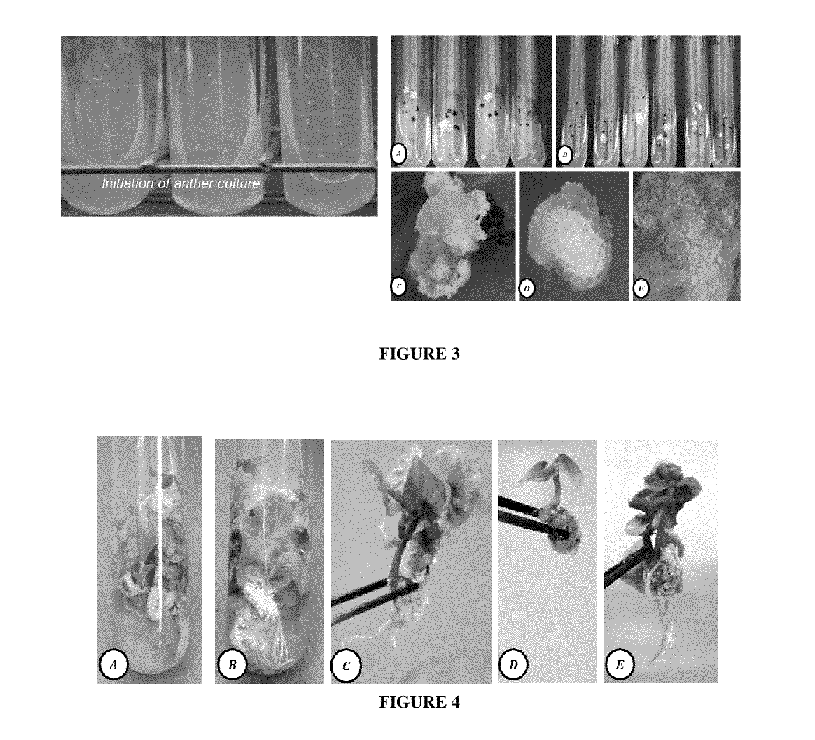

In an embodiment, the anthers are excised from unopened flower buds under aseptic conditions and inoculated on to the composition of the present disclosure for callus induction. FIG. 3 illustrates callus initiation from anthers of Jatropha.

In another embodiment, the process of producing Jatropha plants including but is not limited haploid plant, double haploid plant, tetraploid plant and polyploid plant comprises steps of:
inoculating anther of the Jatropha plant in the composition described above;
inducing callus formation, followed by proliferation; and
inducing somatic embryogenesis, followed by embryo germination, thereby producing Jatropha plants.

In an embodiment, in the process, callus induction was carried out by incubating the composition inoculated with the anther in dark at a temperature ranging from about 24° C. to 26° C. for a period ranging from about 25 days to 30 days.

In another embodiment, in the process, the callus induction was carried out by incubating the composition inoculated with the anther in dark at a temperature of about 24° C., about 24.5° C., about 25° C., about 25.5° C. or about 26° C. for a period of about 25 days, 25.5 days, 26 days, 26.5 days 27 days, 27.5 days, 28 days, 28.5 days, 29 days, 29.5 days or about 30 days.

In an embodiment, in the process, during the callus induction, the composition is replenished at a period of about 10 days to 15 days for high frequency of callus induction.

In another embodiment, in the process, during callus the induction, the composition is replenished at a duration of about 10 days, about 11 days, about 12 days, about 13 days, about 14 days or about 15 days, for high frequency of callus induction.

In an embodiment, the composition in the process, for inducing callus formation comprises MS macro element, MS micro element, vitamin, growth regulator, additive, carbohydrate, binder, buffer, antioxidant, sugar alcohol and gelling agent.

In an embodiment, the composition for the callus induction comprises the MS macro elements selected form a group comprising ammonium nitrate, potassium nitrate, calcium chloride dehydrate, magnesium sulfate heptahydrate and monopotassium phosphate, or any combinations thereof; the MS micro elements selected from a group comprising potassium iodide, dihydrogen borate, manganese sulfate tetrahydrate, zinc sulfate heptahydrate, sodium molybdate dehydrate, copper sulfate pentahydrate, cobalt chloride hexahydrate, ethylene diamine tetraacetic acid disodium salt dihydrate and ferrous sulfate heptahydrate, or any combinations thereof; the vitamins selected from a group comprising inositol, glycine, thiamine, pyridoxine HCl and nicotinic acid, or any combinations thereof; the growth regulator selected from a group comprising 2,4-dichloro phenoxy acetic acid (2,4-D), benzyl amino purine (BA), indole-3-acetic acid (IAA) and kinetin (6-furfurly amino purine), or any combinations thereof; the additive selected from a group comprising casein hydrolysate, coconut milk, Malt extract and yeast extract, or any combinations thereof; the carbohydrate selected from a group comprising maltose, sucrose, glucose and fructose, or any combinations thereof; the binder selected from a group comprising polyvinyl pyrrolidone and charcoal, or a combination thereof; and the buffer selected from a group comprising citric acid, and MES (2-(N-morpholino)ethanesulfonic acid), or a combination thereof; the sugar alcohol selected from a group comprising myo inositol and sorbitol, or a combination thereof; the antioxidant selected from a group comprising ascorbic acid and Dithiothreitol (DTT), or a combination thereof; and the gelling agent selected from a group comprising agar and gellan gum.

In an embodiment, the composition for callus induction comprises the MS macro elements at a concentration ranging from about 10% v/v to 20% v/v with respect to 10×MS macro element stock concentration; the MS micro elements at a concentration ranging from about 1% v/v to 5% v/v with respect to 100×MS micro element stock concentration; the vitamins at a concentration ranging from about 0.5% v/v to 1% v/v with respect to 200×MS vitamin stock concentration; the binder at a concentration ranging from about 0.025% to 0.05% w/v; the buffer at a concentration ranging from about 0.003% to 0.006% w/v; the additive at a concentration ranging from about 0.03% to 0.05% w/v; the carbohydrate at a concentration ranging from about 2% to 9% w/v; growth regulator at a concentration ranging from 0.00001% to 0.0005%; antioxidant at a concentration ranging from about 0.005% to 0.01%; sugar alcohol at a concentration ranging from about 0.01% to 0.02 w/v %; and the gelling agent at a concentration ranging from about 0.3 to 0.75% w/v.

In an embodiment, the composition inducing callus formation comprises MS macro elements, MS micro elements, MS vitamins, citric acid, polyvinyl pyrrolidone, ascorbic acid, myo inositol, maltose, casein hydrdolysate, 2,4-Dichloro phenoxy acetic acid (2,4-D), benzyl amino purine (BA), kinetin (6-furfuryl amino purine) and agar.

In an embodiment, the pH of the composition for inducing callus formation is ranging from about 5.6 to 5.8.

In another embodiment, the pH of the composition for inducing callus formation is about 5.6, about 5.7 or about 5.8.

In an embodiment, after about 25 days to 30 days of callus induction, micro calli is separated and inoculated into the fresh composition of the present disclosure, followed by incubating the inoculated composition in dark for about 21 days to 25 days, for proliferation. The composition is now dedicated as proliferation medium. The micro calli is transferred to the said fresh composition for proliferation and to increase the quantity of calli.

In an embodiment, the composition for the callus proliferation comprises MS macro element, MS micro element, vitamin, growth regulator, carbohydrate, binder, buffer, antioxidant, sugar alcohol and gelling agent.

In an embodiment, the composition for proliferation comprises the MS macro element selected form a group comprising ammonium nitrate, potassium nitrate, calcium chloride dehydrate, magnesium sulfate heptahydrate and monopotassium phosphate, or any combinations thereof; the MS micro element selected from a group comprising potassium iodide, dihydrogen borate, manganese sulfate tetrahydrate, zinc sulfate heptahydrate, sodium molybdate dehydrate, copper sulfate pentahydrate, cobalt chloride hexahydrate, ethylene diamine tetra acetic acid disodium salt dihydrate and ferrous sulfate heptahydrate, or any combinations thereof; the vitamin selected from a group comprising thiamine HCl, pyridoxine HCl and nicotinic acid, or any combinations thereof; the growth regulator selected from a group comprising indole-3-acetic acid (IAA), benzyl amino purine (BA), kinetin (6-furfurly amino purine), or any combinations thereof; the carbohydrate selected from a group comprising sucrose, glucose, maltose and fructose, or any combinations thereof; the binder selected from a group comprising polyvinyl pyrrolidone and charcoal, or a combination thereof; the buffer selected from a group comprising citric acid and MES, or a combination thereof; the sugar alcohol selected from a group comprising myo inositol and sorbitol, or a combination thereof; the antioxidant selected from a group comprising ascorbic acid and DTT, or a combination thereof; and the gelling agent selected from a group comprising agar and gellan gum.

In an embodiment, the composition for proliferation comprises the MS macro elements at a concentration ranging from about 10% v/v to 20% v/v with respect to 10×MS macro element stock concentration; the MS micro elements at a concentration ranging from about 1% v/v to 5% v/v with respect to 100×MS micro element stock concentration; the vitamins at a concentration ranging from about 0.5% v/v to 1% v/v with respect to 200×MS vitamin stock concentration; the binder at a concentration ranging from about 0.025% w/v to 0.05% w/v; the buffer at a concentration ranging from about 0.003% w/v to 0.006% w/v; the carbohydrate at a concentration ranging from about 3% w/v to 6% w/v; growth regulator at a concentration ranging from 0.00001% w/v to 0.0005% w/v; antioxidant at a concentration ranging from about 0.005% w/v to 0.01% w/v; sugar alcohol at a concentration ranging from about 0.01% w/v to 0.02% w/v; and the gelling agent at a concentration ranging from about 0.4% w/v to 0.7% w/v.

In an embodiment, the composition for callus proliferation comprises MS macro elements, MS micro elements, B5 vitamins, citric acid, polyvinyl pyrrolidone, ascorbic acid, myo inositol, sucrose, indole-3-acetic acid (IAA), benzyl amino purine (BA), kinetin (6-furfurly amino purine) and agar.

In an embodiment, the pH of the composition for the proliferation is ranging from about 5.6 to 5.8.

In another embodiment, the pH of the composition for the proliferation is about 5.6, about 5.7 or about 5.8.

In an embodiment, in the process the calli having a size ranging from about 0.5 cm to 1.0 cm during the proliferation are divided into pieces and distributed/inoculated evenly on the composition and incubated in dark at a temperature ranging from about 25° C. to 28° C. for period ranging from about 10 days to 15 days for inducing somatic embryogenesis.

In another embodiment, in the process the calli having a size of about 0.5 cm, about 0.6 cm, about 0.7 cm, about 0.8 cm, about 0.9 cm or about 1.0 cm, during the proliferation are divided into pieces and distributed/inoculated evenly on the composition of the present disclosure and incubated in dark at a temperature of about 25° C., about 26° C., about 27° C. or about 28° C., for a period of about 10 days, about 11 days, about 12 days, about 13 days, about 14 days or about 15 days, for inducing somatic embryogenesis.

In an embodiment, the composition for inducing somatic embryogenesis comprises MS macro element, MS micro element, vitamin, carbohydrate, binder, buffer, antioxidant, sugar alcohol and gelling agent.

In an embodiment, the composition for inducing somatic embryogenesis comprises the MS macro element selected form a group comprising ammonium nitrate, potassium nitrate, calcium chloride dehydrate, magnesium sulfate heptahydrate and monopotassium phosphate, or any combinations thereof; the MS micro element selected from a group comprising potassium iodide, dihydrogen borate, manganese sulfate tetrahydrate, zinc sulfate heptahydrate, sodium molybdate dehydrate, copper sulfate pentahydrate, cobalt chloride hexahydrate, ethylene diamine tetraacetic acid disodium salt dihydrate and ferrous sulfate heptahydrate, or any combinations thereof; the vitamin selected from a group comprising inositol, glycine, thiamine, pyridoxine HCl and nicotinic acid, or any combinations thereof; the carbohydrate selected from a group comprising maltose, glucose, sucrose and fructose, or any combinations thereof; the binder selected from a group comprising polyvinyl pyrrolidone and charcoal or a combination thereof; the buffer selected from a group comprising citric acid, and MES, or a combination thereof; the sugar alcohol selected from a group comprising myo inositol and sorbitol, or a combination thereof; the antioxidant selected from a group comprising ascorbic acid and DTT, or a combination thereof; and the gelling agent selected from a group comprising agar and gellan gum (phytagel).

In an embodiment, the composition for inducing somatic embryogenesis comprises the MS macro elements at a concentration ranging from about 10% v/v to 20% v/v with respect to 10×MS macro element stock concentration; the MS micro elements at a concentration ranging from about 1% v/v to 5% v/v with respect to 100×MS micro element stock concentration; the vitamins at a concentration ranging from about 0.5% v/v to 1% v/v with respect to 200×MS vitamin stock concentration; the binder at a concentration ranging from about 0.025% w/v to 0.05% w/v; the buffer at a concentration ranging from about 0.003% w/v to 0.006% w/v; the carbohydrate at a concentration ranging from about 2% w/v to 3% w/v; antioxidant at a concentration ranging from about 0.005% w/v to 0.01% w/v; sugar alcohol at a concentration ranging from about 0.01% w/v to 0.02% w/v; and the gelling agent at a concentration ranging from about 1% w/v to 10% w/v.

In an embodiment, the composition for inducing somatic embryogenesis comprises MS macro elements, MS micro elements, MS vitamins, citric acid, polyvinyl pyrrolidone, ascorbic acid, myo inositol, maltose and phytagel.

In an embodiment, the pH the composition for inducing somatic embryogenesis is ranging from about 5.6 to 5.8.

In another embodiment, the pH of the composition for inducing somatic embryogenesis is about 5.6, about 5.7 or about 5.8.

In an embodiment, the somatic embryoid developed during somatic embryogenesis is transferred/inoculated to the composition of the present disclosure for somatic embryo maturation, followed by incubating at a temperature ranging from about 24° C. to 26° C. for a period ranging from about 14 hours to 16 hours under light (photoperiod), and followed by incubating at a temperature ranging from about 24° C. to 26° C. for period ranging from about 8 hours to 10 hours, in dark.

In an alternate embodiment, in the process, the somatic embryogenesis is continued until somatic embryos get matured and small shoots are visible.

In an embodiment, the composition for the somatic embryo maturation comprises MS macro elements, MS micro elements, vitamins, growth regulator, additive, carbohydrate, amino acid, binder, buffer, antioxidant, sugar alcohol and gelling agent.

In an embodiment, the composition for somatic embryo maturation comprises the MS macro elements selected form a group comprising ammonium nitrate, potassium nitrate, calcium chloride dehydrate, magnesium sulfate heptahydrate and monopotassium phosphate, or any combinations thereof; the MS micro elements selected from a group comprising potassium iodide, dihydrogen borate, manganese sulfate tetrahydrate, zinc sulfate heptahydrate, sodium molybdate dehydrate, copper sulfate pentahydrate, cobalt chloride hexahydrate, ethylene diamine tetraacetic acid disodium salt dihydrate and ferrous sulfate heptahydrate, or any combinations thereof; the vitamin selected from a group comprising inositol, glycine, thiamine, pyridoxine HCl and nicotinic acid, or any combinations thereof; the growth regulator selected from a group comprising benzyl amino purine (BA), kinetin (6-furfurly amino purine) and indole butyric acid, or any combinations thereof; the additive selected from a group comprising casein hydrolysate, coconut milk, malt extract and yeast extract, or any combinations thereof; the carbohydrate selected from a group comprising maltose, glucose, sucrose and fructose, or any combinations thereof; the binder selected from a group comprising polyvinyl pyrrolidone and charcoal, or a combination thereof; the buffer selected from a group comprising citric acid, and MES, or any combination thereof; the amino acid selected from a group comprising proline, glycine, serine, glutamine and alanine or any combinations thereof; the sugar alcohol selected from a group comprising myo inositol and sorbitol, or a combination thereof; the antioxidant selected from a group comprising ascorbic acid and DTT, or any combination thereof; and the gelling agent selected from a group comprising agar and gellan gum.

In an embodiment, the composition for somatic embryo maturation comprises the MS macro elements at a concentration ranging from about 10% v/v to 20% v/v with respect to 10×MS macro element stock concentration; the MS micro elements at a concentration ranging from about 1% v/v to 5% v/v with respect to 100×MS micro element stock concentration; the vitamins at a concentration ranging from about 0.5% v/v to 1% v/v with respect to 200×MS vitamin stock concentration; the binder at a concentration ranging from about 0.025% w/v to 0.05% w/v; the buffer at a concentration ranging from about 0.003% w/v to 0.006% w/v; the protein at a concentration ranging from about 0.03% w/v to 0.05% w/v; the carbohydrate at a concentration ranging from about 2% w/v to 6% w/v; growth regulator at a concentration ranging from −0.00001% w/v to 0.0005% w/v; antioxidant at a concentration ranging from about 0.005% w/v to 0.01% w/v; sugar alcohol at a concentration ranging from about 0.01% w/v to 0.02% w/v; amino acid at concentration ranging 0.05% w/v to 6% w/v, and the gelling agent at a concentration ranging from about 0.4% w/v to 0.75% w/v.

In an embodiment, the composition for somatic embryo maturation comprises MS macro elements, MS micro elements, MS vitamins, citric acid, polyvinyl pyrrolidone, ascorbic acid, myo inositol, maltose, proline, casein hydrolysate, sorbitol, benzyl amino purine (BA), kinetin (6-furfurly amino purine), indole butyric acid and phytagel.

In an embodiment, the pH of the composition for somatic embryo maturation is ranging from about 5.6 to 5.8.

In another embodiment, the pH of the composition for somatic embryo maturation is about 5.6, about 5.7 or about 5.8.

In an embodiment, the matured embryoids or small plantlets during somatic embryogenesis are transferred/inoculated in to the composition of the present disclosure for embryo germination and for regeneration of plants by proliferation of roots and elongation of shoots.

In an embodiment, the embryogenic calli which are not matured during somatic embryogenesis are sub cultured till the mature embryoids or plantlets are formed.

In an embodiment, the composition of the present disclosure for the somatic embryo germination comprises MS macro elements, MS micro elements, vitamins, growth regulators, carbohydrate, binder, buffer, antioxidant, sugar alcohol and gelling agent.

In an embodiment, the composition for somatic embryo germination comprises the MS macro elements selected form a group comprising ammonium nitrate, potassium nitrate, calcium chloride dehydrate, magnesium sulfate heptahydrate and monopotassium phosphate or any combinations thereof; the MS micro element selected from a group comprising potassium iodide, dihydrogen borate, manganese sulfate tetrahydrate, zinc sulfate heptahydrate, sodium molybdate dehydrate, copper sulfate pentahydrate, cobalt chloride hexahydrate, ethylene diamine tetra acetic acid disodium salt dihydrate and ferrous sulfate heptahydrate, or any combinations thereof; the vitamin selected from a group comprising inositol, glycine, thiamine, pyridoxine HCl and nicotinic acid, or any combinations thereof; the growth regulator selected from a group comprising indole butyric acid, phloroglucinol and gibberellic acid, or any combinations thereof; the carbohydrate selected from a group comprising sucrose, glucose, sucrose and maltose, or any combinations thereof; the binder selected from a group comprising polyvinyl pyrrolidone and charcoal, or a combination thereof; the buffer selected from a group comprising citric acid and MES, or a combination thereof; the sugar alcohol selected from a group comprising myo inositol and sorbitol, or a combination thereof; and the antioxidant selected from a group comprising ascorbic acid and DTT, or a combination thereof; and the gelling agent selected from a group comprising agar and gellan gum.

In an embodiment, the composition for somatic embryo germination comprises the MS macro elements at a concentration ranging from about 10% v/v to 20% v/v with respect to 10×MS macro element stock concentration; the MS micro elements at a concentration ranging from about 1% v/v to 5% v/v with respect to 100×MS micro element stock concentration; the vitamins at a concentration ranging from about 0.5% v/v to 1% v/v with respect to 200×MS vitamin stock concentration; the binder at a concentration ranging from about 0.025% w/v to 0.05% w/v; the buffer at a concentration ranging from about 0.003% w/v to 0.006% w/v; the carbohydrate at a concentration ranging from about 1% w/v to 2% w/v; growth regulator at a concentration ranging from 0.00001% w/v to 0.0002% w/v; antioxidant at a concentration ranging from about 0.005% w/v to 0.01% w/v; sugar alcohol at a concentration ranging from about 0.01% w/v to 0.02% w/v; and the gelling agent at a concentration ranging from about 0.7 w/v to 0.8% w/v.

In an embodiment, the composition for somatic embryo germination comprises MS macro elements, MS micro elements, MS vitamins, citric acid, polyvinyl pyrrolidone, ascorbic acid, myo inositol, sucrose, indole butyric acid, phloroglucinol, gibrellic acid and agar.

In an embodiment, the pH of the composition for somatic embryo germination is ranging from about 5.6 to 5.8.

In another embodiment, the pH of the composition for somatic embryo germination is about 5.6, about 5.7 or about 5.8.

In another embodiment, the regeneration from morphogenic embryogenic calli or embroyoids is stimulated and maintained in various combinations of MS basal medium with B5 vitamins containing growth regulators.

In an embodiment, regenerated shoots upon regeneration are transferred to rooting medium or germination medium for root development.

In an embodiment, the process of developing haploid, double haploid or polyploid plant of Jatropha species by anthers, using the composition described herein is genotype independent and is reproducible in all genotypes of Jatropha species.

In an embodiment, the process of the present disclosure provides for an anther culture system to produce pure homozygous plants of Jatropha species in single generation when compared to breeding techniques available in the art, which takes about 8 generations to 10 generations requiring about 8 years to 10 years.

Acclimatization of the Jatropha Plants developed by the process of the present disclosure—
The fully developed plants/plantlets (with shoots and roots) were removed from the culture tubes and washed in running tap water to remove agar media.
The plants were initially transferred to liquid media (Hoagland media solution) and incubated for a period ranging from about 7 days to 10 days.
Later, the plants were transferred to a glass bottle containing a mixture of sterilized soil rite, followed by incubating under light conditions in a climatic chamber for a period ranging from about 15 days to 20 days.
The fully established plants were subsequently transferred to pots after a period ranging from about 15 days to 20 days of hardening in climatic chamber and moved to green house.
About 80% of the plants developed survived in soil and these plants were phenotypically normal, like the parental plants.

Additional embodiments and features of the present disclosure will be apparent to one of ordinary skill in art based upon the description provided. The embodiments provide various features and advantageous details thereof in the description. Descriptions of well-known/conventional methods and techniques are omitted so as to not unnecessarily obscure the embodiments. The examples provided herein are intended merely to facilitate an understanding of ways in which the embodiments provided may be practiced and to further enable those of skill in the art to practice the embodiments provided. Accordingly, the following examples should not be construed as limiting the scope of the embodiments.

### Examples

### Example 1: Preparation of Media Composition of the Present Disclosure

a. Preparation of Composition for Callus Induction—

About 100 ml of MS macro elements (10× concentration), about 25 ml of MS micro elements (100× concentration), about 10 ml of MS vitamins (200× concentration), about 40 mg of citric acid, about 250 mg of polyvinyl pyrrolidone, about 55 mg of ascorbic acid, about 125 mg of myo inositol, about 45 g of maltose, about 125 mg of casein hydrolysate, about 5 mg of 2,4, dichloro phenoxy acetic acid (2.4-D), about 1 mg of benzyl amino purine (BA) and about 3 mg of kinetin (6-furfuryl amino purine) are dissolved in MilliQ water to obtain a solution. The pH of the solution is adjusted to 5.6 by adding potassium hydroxide. The solution is made up to the volume of 1000 ml with milliQ, followed by adding about 7.5 g of agar and autoclaved at a temperature of about 121° C. to obtain the sterile medium composition.

b. Preparation of Composition for Proliferation—

About 100 ml of MS macro elements (10× concentration), about 25 ml of MS micro elements (100× concentration) about 10 ml of B5 vitamins (200× concentration), about 40 mg of citric acid, about 250 mg of polyvinyl pyrrolidone, about 55 mg of ascorbic acid, about 125 mg of myo inositol, about 60 g of sucrose, about 125 mg of casein hydrolysate, about 4 mg of indole 3 acetic acid, about 1 mg of benzyl amino purine (BA) and about 3 mg of kinetin (6-furfuryl amino purine) are dissolved in MilliQ water to obtain a solution. The pH of the solution is adjusted to 5.8 by adding potassium hydroxide. The solution is made up to the volume of 1000 ml with milliQ, followed by adding about 7 g of agar, to obtain the composition.

c. Preparation of Composition for Somatic Embryo Induction—

About 100 ml of MS macro elements (10× concentration), about 25 ml of MS micro elements (100× concentration) about 10 ml of MS vitamins (200× concentration), about 40 mg of citric acid, about 250 mg of polyvinyl pyrrolidone, about 55 mg of ascorbic acid, about 100 mg of myo inositol and about 25 g of maltose are dissolved in MilliQ water, to obtain a solution. The pH of the solution is adjusted to 5.8 by adding potassium hydroxide. The solution is made up to the volume of 1000 ml with milliQ, followed by adding about 10 g of phytagel, to obtain the composition.

d. Preparation of Composition for Somatic Embryo Maturation—

About 100 ml of MS macro elements (10× concentration), about 25 ml of MS micro elements (100× concentration) about 10 ml of MS vitamins (200× concentration), about 40 mg of citric acid, about 250 mg of polyvinyl pyrrolidone, about 55 mg of ascorbic acid, about 100 mg of myo inositol, about 25 g of maltose, about 300 g of proline, about 500 mg of casein hydrolysate, about 30 g of sorbitol, about 1 mg of benzyl amino purine (BA) and about 0.5 mg of kinetin (6-furfuryl amino purine) and about 0.2 mg of indole butyric acid, are dissolved in MilliQ water to obtain a solution. The pH of the solution is adjusted to 5.8 by adding potassium hydroxide. The solution is made up to the volume of 1000 ml with milliQ, followed by adding about 4 g of phytagel, to obtain the composition.

e. Preparation of Composition for Embryo Germination—

About 100 ml of MS macro elements (10× concentration), about 25 ml of MS micro elements (100× concentration) about 10 ml of MS vitamins (200× concentration)n, about 40 mg of citric acid, about 250 mg of polyvinyl pyrrolidone, about 55 mg of ascorbic acid, about 100 mg of myo inositol, about 20 g of sucrose, about 300 g of proline, about 500 mg of casein hydrolysate, about 30 g of sorbitol, about 0.1 mg of indole butyric acid, about 0.25 mg of phloroglucinol and about 0.5 mg of gibrellic acid are dissolved in MilliQ water to obtain a solution. The pH of the solution is adjusted to 5.8 by adding potassium hydroxide. The solution is made up to the volume of 1000 ml with milliQ, followed by adding about 8 g of agar, to obtain the composition.

### Example 2: Process of Producing Jatropha Plants Employing the Composition of the Present Disclosure

a. Surface Sterilization of Flower Buds
Healthy and disease-free inflorescence of Jatropha lines were collected;
The inflorescence (containing flower buds) were thoroughly washed in running tap water and then treated with water containing about 25 μl-50 μl of detergent solution;
The flower buds were thoroughly washed in running tap water for a period ranging from about 5 minutes to 10 minutes;
The flower buds were treated with 0.1% Bavistin in laminar air flow chamber and washed in milliQ water for about 4 times to 5 times.
The inflorescence (containing flower buds) were surface sterilized by immersing in 70% (v/v) ethanol for a period ranging from about 1 minute to 3 minutes. Thereafter, the inflorescence is sterilized with 50% (v/v) sodium hypochlorite for about 15 minutes containing about 50 μl of tween 20 and rinsed in sterile water for about 5 times to 6 times, to completely remove foam.
The inflorescence is then sterilized with 50% (v/v) sodium hypochlorite for about 15 minutes without tween 20 and rinsed in sterile water for about 5 times to 6 times.
The inflorescence was then incubated at a temperature of about 4° C. for pretreatment for a period of about 4 days to 7 days in a closed container under aseptic condition.

b. Callus Induction and Proliferation

The flower bud of about 2.5 mm were selected for excision of anthers. The flower buds were blot dried with several layers of sterile filter papers to remove excess moisture.

The anthers were excised from unopened flower buds in the laminar air flow (sterile hood) under aseptic conditions.

About 10 to 12 anthers were inoculated into culture tubes comprising the composition of the present disclosure for callus induction. The inoculated culture tubes were incubated in dark at a temperature of about 25±1° C. for a period ranging from about 25 days to 30 days. The culture tubes were replenished with the composition every three weeks for high frequency induction of callus.

After about 25 days to 30 days, the micro calli were separated and inoculated into the composition of the present disclosure for proliferation. The inoculated composition was incubated in dark for a period ranging from about 21 days to 25 days.

The nature and morphology of calli from anthers formed on different media and for different genotype were observed every 15 days.

Table 1 illustrates the callus induction (callus formation) in the composition comprising 100 ml of MS macro elements (10× concentration), about 25 ml of MS micro elements (100× concentration) about 10 ml of MS vitamins (200× concentration), citric acid—40 mg/l; PVP—250 mg/l; Ascorbic acid—55 mg/l; myo inositol—125/L; Maltose—45 g/l; Casein hydro lysate—125 mg/1; 2-4-D—5 mg/l (or Dicamba 2 mg/l); Benzyl amino purine (BA)—1 mg/l; Kinetin (6-furfuryl amino purine)—3 mg/l and Agar—7.5 g/l.

TABLE 1

| | Concentration | |
|---|---|---|
| Growth Regulators | (mg/L) | Callus formation (%) |
| 2,4-D | 0.5 | 2 |
| | 1 | 2 |
| | 2 | 10 |
| | 3 | 20 |
| | 4 | 35 |
| | 5 | 45 |
| 2,4 D + BAP | 5.0 + 0.5 | 50 |
| | 5.0 + 1.0 | 65 |
| | 5.0 + 2.0 | 60 |
| | 5.0 + 3.0 | 60 |
| | 5.0 + 4.0 | 55 |
| | 5.0 + 5.0 | 40 |
| 2,4 D + BAP + | 5.0 + 1.0 + 0.5 | 70 |
| Kinetin | | |
| | 5.0 + 1.0 + 1.0 | 80 |
| | 5.0 + 1.0 + 2.0 | 85 |
| | 5.0 + 1.0 + 3.0 | 100 |
| | 5.0 + 1.0 + 4.0 | 80 |
| | 5.0 + 1.0 + 5.0 | 45 |

Table 2 illustrates the effect of different concentration of IAA individually and in combination with BAP and Kinetin of different concentrations on callus proliferation. Among the different concentration tested, IAA—4 mg/l; Benzyl amino purine (BA)—1 mg/l; Kinetin (6-furfuryl amino purine) 3 mg/l was found to be highly effective in producing high quantity of callus in a composition of medium comprising. 100 ml of MS macro elements (10× concentration), about 25 ml of MS micro elements (100× concentration) about 10 ml of B5 vitamins (200× concentration); citric acid—40 mg/l; PVP—250 mg/l; Ascorbic acid—55 mg/l; myoinositol—125/L; Sucrose—60 g/l; Casein hydrolysate—100 mg/l; Agar—7 g/l.

TABLE 2

| Growth | Concentration | |
|---|---|---|
| Regulators | (mg/L) | Callus quantity |
| IAA | 0.5 | + |
| | 1.0 | + |
| | 2.0 | + |
| | 3.0 | + |
| | 4.0 | ++ |
| | 5.0 | + |
| IAA + BAP | 4.0 + 0.5 | ++ |
| | 4.0 + 1.0 | +++ |
| | 4.0 + 2.0 | ++ |
| | 4.0 + 3.0 | ++ |
| | 4.0 + 4.0 | + |
| | 4.0 + 5.0 | + |
| IAA + BAP + | 4.0 + 1.0 + 0.5 | +++ |
| Kin | | |
| | 4.0 + 1.0 + 1.0 | +++ |
| | 4.0 + 1.0 + 2.0 | +++ |
| | 4.0 + 1.0 + 3.0 | +++++ |
| | 4.0 + 1.0 + 4.0 | +++ |
| | 4.0 + 1.0 + 5.0 | ++ |

c. Induction of Somatic Embryogenesis and Germination.

The calli of size ranging from about 0.5 cm to 1.0 cm (breadth) were divided into small pieces and inoculated in the culture tubes comprising the composition of the present disclosure for induction of somatic embryogenesis.

The culture tubes showing the developed somatic embryoids were transferred to composition of the present disclosure for somatic embryo maturation and incubated in light culture room for about 16 hours under photoperiod (provided by cool while fluorescent lamp) and under dark for about 8 hours at a temperature of about 25±1° C. This process is continued until somatic embryos get matured and small shoots are visible.

The matured embryoids with the photoperiod of 16 hrs light and 8 hrs dark or small plantlets were transferred to the composition of the present disclosure and incubated at temperature ranging from about 25° C. to 28° C. for period of about 25 days to 30 days, for germination, for proliferation of roots and further elongation of shoots.

The embryogenic calli which were not matured were further sub cultured till mature embryoids or plantlets are obtained.

Regeneration of embryogenic calli or embryoids was stimulated and maintained on various combinations of MSB media. Regenerated shoots were transferred to rooting medium for root development.

Table 3 illustrates the effect of different concentration of IBA individually and in combination with BAP and Kinetin of different concentrations on plant regeneration from embryogenic calli of Jatropha anthers. Among the different concentration tested, IBA—0.1 mg/l, Benzyl amino purine (BA)—1 mg/l; Kinetin (6-furfuryl amino purine) 0.5 mg/l was found to be highly effective in producing more number shoots (3 shoots/embryogenic calli) in 20% of the cultures employing the composition comprising 100 ml of MS macro elements (10× concentration), about 25 ml of MS micro elements (100× concentration) about 10 ml of MS vitamins (200× concentration); citric acid—40 mg/l; PVP—250 mg/l; Ascorbic acid—55 mg/l; myo inositol—100/L; Maltose—25 g/l; Casein hydro lysate—500 mg, Sorbitol 30 g/l; proline 300 mg/l, Phytagel 4 g/l.

TABLE 3

| Growth | Concentration | % of cultures showed | No of Plants/ |
|---|---|---|---|
| Regulators | (mg/L) | regeneration | explant |
| IBA | 0 | 0.0 | 0 |
| | 0.1 | 0.0 | 0 |
| | 0.2 | 0.0 | 0 |
| | 1.0 | 0.0 | 0 |
| | 2.0 | 0.0 | 0 |
| | 5.0 | 0.0 | 0 |
| IBA + BAP | 0.1 + 0.5 | 0.0 | 2 |
| | 0.1 + 1.0 | 10.0 | |
| | 0.1 + 2.0 | 0.0 | 0 |
| | 0.1 + 3.0 | 0.0 | 0 |
| | 0.1 + 4.0 | 0.0 | |
| | 0.5 + 5.0 | 0.0 | 0 |
| IBA + BAP + | 0.1 + 1.0 + 0.5 | 20.0 | 3 |
| Kin | | | |
| | 0.1 + 1.0 + 1.0 | 15.0 | 1 |
| | 0.1 + 1.0 + 2.0 | 0.0 | 0 |
| | 0.1 + 1.0 + 3.0 | 0.0 | 0 |
| | 0.1 + 1.0 + 4.0 | 0.0 | 0 |
| | 0.1 + 1.0 + 5.0 | 0.0 | 0 |

Table 4 illustrates the effect of different concentrations of IBA individually and in combination of with various concentrations of phloroglucinol and GA3 on embryo germination and rooting of seedling. Among the tested concentrations, IBA-0.1 mg/l, phloroglucinol—0.5 mg/l; Gibberellic acid-0.5 mg/l observed to be effective in producing more number of roots (6 roots/shoot) in 80% of the cultures; employing the composition comprising 100 ml of MS macro elements (10× concentration), about 25 ml of MS micro elements (100× concentration) about 10 ml of MS vitamins (200× concentration); citric aicd—40 mg/l; PVP—250 mg/l; Ascorbic acid—55 mg/l; myo inositol—100 mg/L; sucrose 20 g/L. Phytagel 1.5 g/l.

TABLE 4

| Growth | Concentration | % of Embryos | No. of roots/ |
|---|---|---|---|
| Regulators | (mg/L) | Germinated | shoot |
| IBA | 0 | 0.0 | 0 |
| | 0.1 | 0.0 | 0 |
| | 0.2 | 0.0 | 0 |
| | 1.0 | 0.0 | 0 |
| | 2.0 | 0.0 | 0 |
| | 5.0 | 0.0 | 0 |
| IBA + | 0.1 + 0.25 | 25.0 | 2 |
| Phloroglucinol | | | |
| | 0.1 + 1.0 | 0.0 | 0 |
| | 0.1 + 2.0 | 0.0 | 0 |
| | 0.1 + 3.0 | 0.0 | 0 |
| | 0.1 + 4.0 | 0.0 | |
| | 0.5 + 5.0 | 0.0 | 0 |
| IBA + | 0.1 + 0.25 + 0.5 | 80.0 | 6 |
| Phloroglucinol + | | | |
| GA3 | | | |
| | 0.1 + 1.0 + 1.0 | 15.0 | 1 |
| | 0.1 + 1.0 + 2.0 | 0.0 | 0 |
| | 0.1 + 1.0 + 3.0 | 0.0 | 0 |
| | 0.1 + 1.0 + 4.0 | 0.0 | 0 |
| | 0.1 + 1.0 + 5.0 | 0.0 | 0 |

### Example 3: Ploidy Analysis of Somatic Embryos/Calli/Regenerated Plants

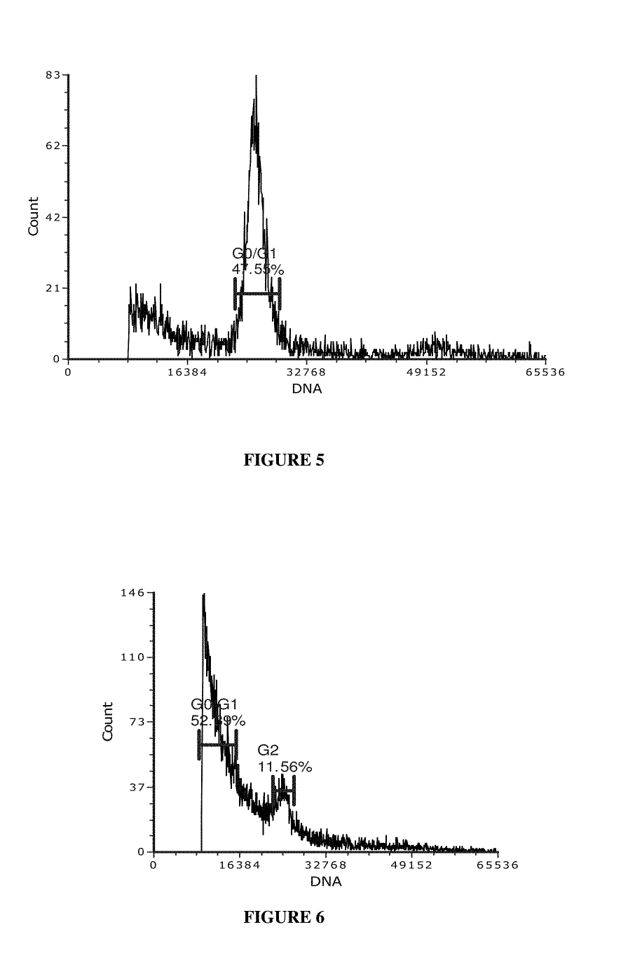

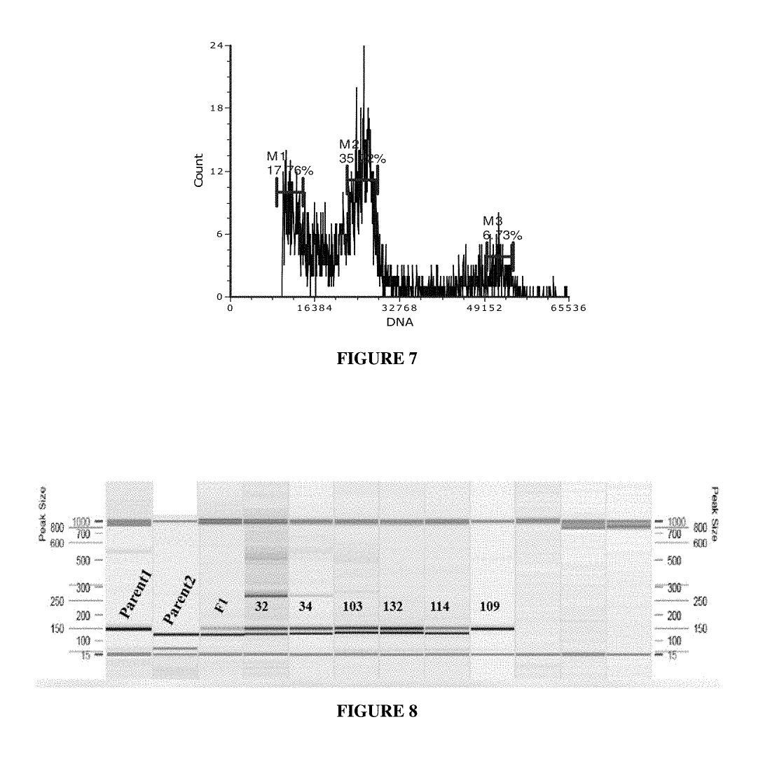

In this experiment, flow cytometry was used to determine the nuclear content of somatic embryos/calli and regenerated plants. The method followed for determination of ploidy is based on the report of Dolezel et al (2007) and the protocol includes chopping of about 200 mg to 300 mg of anther derived embryogenic calli or young leaves of plants (as per the experiment of Example 2) in about 0.4 ml of NE Buffer and the crude extract was filtered with about 30μ filter after incubation for about 15 minutes in Ice. Filtrate is then stained with propidium iodide based staining solution supplied by kit CyStain® UV Precise P for about 10 minutes. The analysis of the nuclei suspension was performed using Sysmex Partec GmbH ploidy analyser. The gate configuration in the instrument set for slow measurement of nuclei (20 nuclei/second) resulting in low variation, around 10000 nuclei measured per sample. The measurement of fluorescence from the nuclei was used in development of histogram. These histograms displayed as DNA peak corresponding to G1 & G2 and calculated DNA of the ploidy of the fluorescence of the samples (embryonic calli and young leaf tissue of the plants) using the following formula: known ploidy control×median position of G1 peak of unknown samples/median position of G1 peak known samples. Flow cytometric analysis of control diploid sample of Jatropha from plant growing in field showed Go/G1 peak at a median value 25717.00. The configuration of this sample has been used for further analysis of the samples such as embryonic calli and young leaf tissue of plants. The flow cytometric analysis of twenty Jatropha embryogenic calli and thirty plants showed clear haploid peak in about 10% to 12% of the samples (illustrated in FIG. 6) which showed DNA content almost half that of control diploid plant (illustrated in FIG. 5). Also, few samples showed peak both at n (median—11788.00) and 2n position (median 25703.50) indicating mixture of haploid and diploid callus or plants.

Further, the leaf samples were collected from two parents and from two parents and from putative haploid and double haploid embryos or leave tissues of plants obtained from example 2. The DNA was extracted, and PCR analysis was carried out with SSR markers for the verification of haploid and dihaploid samples. A total of about 10 polymorphic SSR markers were used on the parents, F1 hybrid and embryogenic calli or tissues of plants. Of the 10 markers, four of them clearly discriminated between parents, F1 and diploid plants and observed spontaneous induction of haploid and dihaploid in about 1% to 2% of the test samples (calli/plants) (see sample 109 in FIG. 8).

Additional embodiments and features of the present disclosure is apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein. The foregoing description of the specific embodiments fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application. While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A composition comprising MS macro element, MS micro element, vitamin, binder, buffer, carbohydrate, sugar alcohol, antioxidant, gelling agent, optionally along with component selected from a group comprising growth regulator, additive and amino, or any combinations thereof.

2. The composition as claimed in claim 1, wherein—
the MS macro element is selected from a group comprising ammonium nitrate, potassium nitrate, calcium chloride dehydrate, magnesium sulfate heptahydrate and monopotassium phosphate, or any combinations thereof;
the MS micro element is selected from a group comprising potassium iodide, dihydrogen borate, manganese sulfate tetrahydrate, zinc sulfate heptahydrate, sodium molybdate dehydrate, copper sulfate pentahydrate, cobalt chloride hexahydrate, ethylene diamine tetra acetic acid disodium salt dihydrate and ferrous sulfate heptahydrate, or any combinations thereof;
the vitamin is selected from a group comprising inositol, thiamine, pyridoxine HCl and nicotinic acid, or any combinations thereof;
the binder is selected from a group comprising polyvinyl pyrrolidone (PVP), and charcoal, or a combination thereof;
the buffer is selected from a group comprising citric acid, and MES (2-(N-morpholino) ethanesulfonic acid), or a combination thereof;
the carbohydrate is selected from a group comprising maltose, sucrose, glucose and fructose, or any combinations thereof;
the sugar alcohol is selected from a group comprising sorbitol and myo-inositol, or a combination thereof;
the antioxidant is selected from a group comprising ascorbic acid and dithiothreitol (DTT), or a combination thereof;
the gelling agent is selected from a group comprising agar and gellan gum, or a combination thereof;
the growth regulator is selected from a group comprising 2,4-dichloro phenoxy acetic acid (2,4-D), benzyl amino purine (BA), kinetin (6-furfuryl amino purine), indole-3-acetic acid, indole butyric acid, phloroglucinol and gibberellic acid, or any combinations thereof;
the additive is selected from a group comprising casein hydrolysate, coconut milk malt extract and yeast extract, or any combination thereof; and
the amino acid is selected from a group comprising proline, glycine, serine, glutamine and alanine, or any combinations thereof;

3. The composition as claimed in claim 1, wherein—
the MS macro elements is at a concentration ranging from about 10% v/v to 20% v/v with respect to 10×MS macro element stock concentration;
the MS micro elements is at a concentration ranging from about 1% v/v to 5% v/v with respect to 100×MS micro element stock concentration;
the vitamins is at a concentration ranging from about 0.5% v/v to 1% v/v with respect to 200×MS vitamin stock concentration;
the binder is at a concentration ranging from about 0.025% w/v to 0.05% w/v;
the buffer is at a concentration ranging from about 0.003% w/v to 0.006% w/v;
the carbohydrate is at a concentration ranging from about 1% w/v to 9% w/v;
the sugar alcohol is at a concentration ranging from about 0.01% w/v to 0.04% w/v;
the antioxidant is at a concentration ranging from about 0.005% w/v to 0.02% w/v;
the gelling agent is at a concentration ranging from about 0.4% w/v to 1% w/v;
the growth regulator is at a concentration ranging from about 0.00001% w/v to 0.0005% w/v;
the additive is at a concentration ranging from about 0.03% w/v to 0.05% w/v; and
the amino acid is at a concentration ranging from about 0.05% w/v to 0.1% w/v;

4. The composition as claimed in claim 1, wherein the composition comprises MS macro element, MS micro element, MS vitamin, citric acid, Polyvinyl pyrrolidone, ascorbic acid, myo inositol, maltose, casein hydrdolysate, 2,4-Dichloro phenoxy acetic acid (2,4-D), benzyl amino purine (BA), kinetin (6-furfuryl amino purine) and agar.

5. The composition as claimed in claim 1, wherein the composition comprises MS macro element, MS micro element, B5 vitamin, citric acid, polyvinyl pyrrolidone, ascorbic acid, myo inositol, sucrose, indole-3-acetic acid (IAA), benzyl amino purine (BA), kinetin (6-furfurly amino purine) and agar.

6. The composition as claimed in claim 1, wherein the composition comprises MS macro elements, MS micro elements, MS vitamins, citric acid, polyvinyl pyrrolidone, ascorbic acid, myo inositol, maltose and phytagel.

7. The composition as claimed in claim 1, wherein the composition comprises MS macro elements, MS micro elements, MS vitamins, citric acid, polyvinyl pyrrolidone, ascorbic acid, myo inositol, maltose, proline, casein hydrolysate, sorbitol, benzyl amino purine (BA), kinetin (6-furfurly amino purine), indole butyric acid and phytagel.

8. The composition as claimed in claim 1, wherein the composition comprises MS macro elements, MS micro elements, MS vitamins, citric acid, polyvinyl pyrrolidone, ascorbic acid, myo inositol, sucrose, indole butyric acid, phloroglucinol, gibrellic acid and agar.

9. A process of preparing the composition as claimed in claim 1 comprises—
dissolving MS macro elements, MS micro elements, vitamins, buffer, binder, antioxidant, sugar alcohol and carbohydrate, optionally along with component selected from a group comprising growth regulator, additive and amino acid, or a combination thereof, in milliQ water; and
adjusting the pH with potassium hydroxide, followed by adding gelling agent to obtain the composition.

10. A process of producing Jatropha plant by employing the composition as claimed in claim 1, said process comprises—
inoculating anther of the Jatropha plant in the composition;
inducing callus formation, followed by proliferation; and
inducing somatic embryogenesis, followed by embryo germination, thereby producing Jatropha plant.

11. The process as claimed in claim 10, wherein the callus formation is induced by incubating the composition comprising anther in dark at a temperature ranging from about 24° C. to 26° C. for a period ranging from about 25 days to 30 days; and wherein during the callus induction, the composition is replenished at a period of about 10 days to 15 days.

12. The process as claimed in claim 10, wherein the callus formation is induced by the composition comprising MS macro elements, MS micro elements, MS vitamins, citric acid, polyvinyl pyrrolidone, ascorbic acid, myo inositol, maltose, casein hydrdolysate, 2,4-Dichloro phenoxy acetic acid (2,4-D), benzyl amino purine (BA), kinetin (6-furfuryl amino purine) and agar.

13. The process as claimed in claim 10, wherein the callus proliferation is carried out by inoculating micro calli into the composition, followed by incubating the composition in dark at temperature ranging from about 25° C. to 27° C. for a period ranging from about 21 days to 25 days.

14. The process as claimed in claim 13, wherein the composition for the callus proliferation comprises MS macro elements, MS micro elements, B5 vitamins, citric acid, polyvinyl pyrrolidone, ascorbic acid, myo inositol, sucrose, indole-3-acetic acid (IAA), benzyl amino purine (BA), kinetin (6-furfurly amino purine) and agar.

15. The process as claimed in claim 10, wherein the somatic embryogenesis is induced by inoculating calli having size ranging from about 0.5 cm to 1.0 cm into the composition, followed by incubating the composition in dark at a temperature ranging from about 25° C. to 28° C. for period ranging from about 10 days to 15 days.

16. The process as claimed in claim 15, wherein the composition for inducing somatic embryogenesis comprises MS macro elements, MS micro elements, MS vitamins, citric acid, polyvinyl pyrrolidone, ascorbic acid, myo inositol, maltose and phytagel.

17. The process as claimed in claim 10, wherein somatic embryoid developed during the somatic embryogenesis is inoculated into the composition, followed by incubating the composition at a temperature ranging from about 24° C. to 26° C. for a period ranging from about 14 hours to 16 hours under light and followed by incubating in dark for a period ranging from about 8 hours to 10 hours, for somatic embryo maturation.

18. The process claimed in claim 17, wherein the composition for the somatic embryo maturation comprises MS macro elements, MS micro elements, MS vitamins, citric acid, polyvinyl pyrrolidone, ascorbic acid, myo inositol, maltose, proline, casein hydrolysate, sorbitol, benzyl amino purine (BA), kinetin (6-furfurly amino purine), indole butyric acid and phytagel.

19. The process as claimed in claim 10, wherein matured embryoids or plantlets are inoculated into the composition and incubated at a temperature ranging from about 25° C. to 27° C. for a period of about 25 days to 30 days for the embryo germination and proliferation of roots and elongation of shoots.

20. The process as claimed in claim 19, wherein the composition for embryo germination comprises MS macro elements, MS micro elements, MS vitamins, citric acid, polyvinyl pyrrolidone, ascorbic acid, myo inositol, sucrose, indole butyric acid, phloroglucinol, gibrellic acid and agar.

21. The process as claimed in claim 10, wherein the pH of the composition is ranging from about 5.6 to 5.8.

22. The process as claimed in claim 10, wherein the process produces haploid Jatropha plant or double haploid Jatropha plant.
